(19) United States
(12) Patent Application Publication
(10) Pub. No.: US 2006/0183980 A1
(43) Pub. Date: Aug. 17, 2006

(54) Mental and physical health status monitoring, analyze and automatic follow up methods and its application on clothing

(76) Inventor: Chang-Ming Yang, Miaoli (TW)

Correspondence Address: HDSL, 4331 STEVENS BATTLE LANE, FAIRFAX, VA, 22033, US

(21) Appl. No.: 11/056,227

(22) Filed: Feb. 14, 2005

(51) Int. Cl. A61B 5/00; A61B 5/02; A61B 5/08; A61B 5/04

(52) U.S. Cl. 600/301; 600/300; 128/905; 128/920; 600/546; 600/500; 600/529

(57) ABSTRACT

A physical and mental health status monitor, analyze and follow up clothing system. Whose purpose is to monitor the user's body fat distribution, muscular activity and position changes to diagnose health status. Regularly giving suggestions to the user to achieve proper exercise and posture to reduce body fat and prevent muscular atrophy. While correcting the user's life style to promote better health, this system also works to reduce mental and emotional stress. Providing better healthcare and reducing the chance of disease. This system includes sensors, a close range monitoring system and long range monitoring stations, through the sensors it collects data for the monitoring system and monitoring stations to observe, analyze, diagnose and follow up on the user's physical and mental state. This system also allows the user and the long range monitoring stations to communicate with each other through the close range monitoring system.

### BACKGROUND OF THE INVENTION

1. Field of the Invention

This invention involves a type of automated monitoring and a diagnostic system for the mental and physical health status of the patient. It refers especially to a type of close and long range monitor, diagnosis, control, and response functions to maintain a continuous two-way monitoring system for the user's mental and physical health. This also means it can provide the user medical advice to promote personal health.

2. Description of the Prior Art

As times change, people live more comfortably and demand a higher standard of living. Due to the advancement in the medical field, human life expectancy is increasing.

On the other hand, in order to pursue more material goods, everyday life has become busier. Due to differing life styles, living habits change regularly to adapt and incorrect living habits can cause many people to not eat, sleep, and function properly. Many people experience too much pressure and are easily affected by their surroundings; these people are easily aggravated, can't relax easily, eat irregularly, are over worked, sleep during the day and lack exercise. All these things lead to poor physical and mental health and the person often leads an unhappy life.

Consequently, building an enriching environment for further advance in the psychological and material worlds has become an important field of study for human health.

In the daily life of an average person, his or her behavior will not exceed the emotional and physical boundaries, but day after day, these changes in behavior can affect that person's health. This invention uses this basis and records down the emotional and physical changes for a continuous long term period. Using close range automated systems; it continuously gathers information to diagnose the user's mental and physical status. It automatically gives advice and continuously checks for improving status. If necessary, this system can also contact the monitoring station for further disease prevention and health care issues.

### SUMMARY OF THE INVENTION

Because everyone's living style changes with a regular pattern, this invention's goal is to provide a clothing system that monitors physical and mental health and automatically analyzes, diagnoses, and provides solutions for any health problems that occur. Through continuous monitoring, analysis and diagnosis of the user's health status, it provides the user with proper health advice to achieve better personal health.

This invention includes but is not limited to the following:
a) Sensors, placed on the garment to acquire physical health data.
b) A close range monitoring system with a microprocessor to receive, analyze, store and transfer data from the sensors or long range monitoring station, and using pictures, voice, images, words, feel, vibrations, 3D projections or drawing software (such as digital white board and paint) or other VR software to provide proper personal information. Based on the result of the diagnosis it provides repeated follow ups.
c) A long range monitoring station, to receive data from the close range monitoring system and provide the average health status for comparison.
d) A mental profile located in the close-range monitoring system. This system will first require the user to move into different positions to acquire muscular information of the user. After analyzing the information, if any muscular irregularities are found (i.e. EMG value too low signifying lack of exercise and suggest more exercise, or EMG too high at rest signifying over reactive muscles and suggest certain relaxation techniques to relax the muscle.)

This invention's method for monitoring first involves the analysis of human behavior, placing sensors in selected areas of the garment, and regularly monitoring the behavioral changes in these areas. Because muscles have a standard structure and limited range for movement, improper actions and movement can lead to improper use of muscles and cause poor health and limit bodily growth. Although modern medicine has already established proper actions and behavior guidelines that benefits personal health, it is difficult to pinpoint different regions of the body and provide proper advice. Through this invention's methods, we can monitor specific regions and record daily behavior, and then based on this data, provide proper suggestions for long-term correction, thus improving the user's health.

At the same time, this invention also monitors body fat distribution in the body. Since food consumption quality has improved, fat and calorie intake is abnormally high. When combined with lack of exercise or improper exercise, this can lead to irregular distribution of body fat causing obesity or eating disorders. These irregularities and disorders further lower a person's immunity, causing more sickness such as diabetes, heart problems, high cholesterol, et cetera. Using this invention, the person can know in advance his or her own body fat distribution and through proper guidance, exercise the proper areas and burn the proper amount of calories or through the correct nutrition correct and control body figure.

Other important factors affecting personal health today are psychological and emotional. Due to busy lifestyles, many people cannot withstand the stress, causing uncontrolled emotions leading to depression, insomnia and other disorders. This invention can monitor the emotional change of the user through the user's tone of voice, focus, change of heart rate, blood pressure and breathing patterns to analyze the person's mood and suggest proper relaxation methods to achieve medical health.

Furthermore, this invention can be used to monitor pregnant mothers, elderly, diabetic, over weight, depressed, blind, deaf, and people suffering from eating disorders, memory loss, and even smoking. This invention provides exercise guidelines to promote physical health. This invention also provides emergency assistance when the need arises to prevent lose of life.

### BRIEF DESCRIPTION OF THE DRAWINGS

FIG. 1 is a systems overview of the current invention;

FIG. 2 is a systems flowchart of the current invention;

FIG. 3 is the standard procedure that the current invention follows;

FIG. 4 is the emergency procedure that the current invention follows; and

FIG. 5 is the procedure the current invention follows for emotional control procedure.

### DETAILED DESCRIPTION OF THE PREFERRED EMBODIMENT

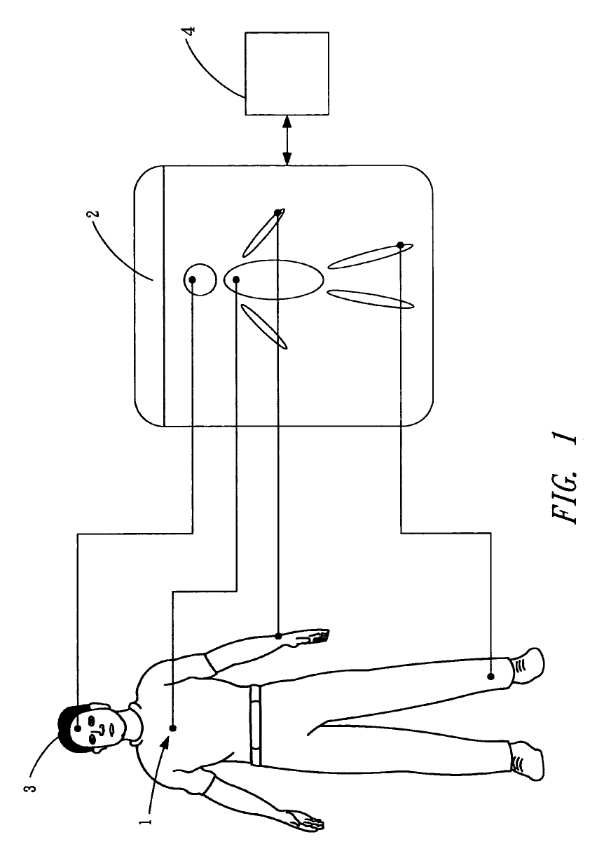

As FIG. 1 show, this invention installs sensor (1), which has many different functions, into the garment of the user. Sensor (1) is removable and placed directly into the user's garment, and can be implanted into the user's body. Sensor 1 can be stationary or mobile, and can move under the control of the user or the close range monitoring system. If necessary, sensor 1 can be removed to clean the garment. This system allows the user (3) to wear the garment for monitoring purposes (when necessary, sensor (1) can be placed within hats, masks, gloves, socks or belts to monitor other areas). When the user has to remove these garments for various situations, sensor (1) can be placed in other locations such as bathtubs, mattresses, toilets, wheelchairs, etc. Sensor (1) includes a range of different sensors such as the Galvanic Skin Reflex sensor, EMG, EKG, breathing sensors, temperature sensors, cameras, body position sensor, mercury switch array, pressure sensors, bend sensors, accelerometer, heat flux sensor, blood oxygen concentration sensors, blood pressure sensors, ultra sound sensors, fetal status sensors, cholesterol sensors, et cetera. These sensors perform the function of monitoring the user's position, fat distribution, breathing rate, fluid distribution, temperature change, heart rate, heat flow and various other functions. Many of these functions do not require extreme modification to sensor (1). For example, measuring the electrical resistance between two points on the body can determine the body fat and fluid distribution. At the same time, the sensors can determine if various muscles are atrophied by performing electric shocks or TENS. In the case, when an abnormality is discovered, the system provides guidance for rehabilitation and follows up on the issue. If no improvements are made, the system can signal the long range monitoring station for two-way communication. This system can also have light sources installed into the garment and have these light sources display words or pictures, for example HELP-BP(200)-Fever(38c), to help alert the user and others.

Barcodes can be installed onto items that the user comes in contact with often, such as the fridge, toilet, television, door, food, cloth and various other items. Then by installing a sensor to read these barcodes, information could be gathered about the frequency that the user uses such items. Electronic tags can be used in place of the barcodes and a radio frequency identification system can be used in place of the barcode reader. By analyzing this data; for example, how many times a day the user comes into contact with the fridge, we can know if the user has abnormal eating habits that could be harmful to his/her health. This invention also allows for sensors to be installed on the walls of the user's home, these sensors may include camera (CCD or CMOS ) or heat imaging sensors to determine the user's position at home. The camera and heat imaging sensors could be allowed to move along grids in the wall to follow the user. If the user is in an abnormal position in the home for an abnormal duration of time, for example, showering for 2 hours, then other sensors could be activated to check on the user's status.

The data collected by sensor (1) as stated above are transmitted to the close range monitoring system (2). The close-range monitoring system (2) can be placed directly inside the garment or be separately placed elsewhere (for example: PDA, cellular phone, computer, et cetera). The system can also include functions such as GPS, LEOS (Low earth orbit system), GPRS (General Packet Radio Service), 3G (Third generation mobile signal transmission), ECDS (An instant image transferal service), MMS (Multi-media service), Blue Tooth, GSM (Global system for mobile communication), CDMA (Code division multiple access), or MPS (mobile positioning system). If the close-range monitoring system is placed apart from the garment, then people living in the same environment can share or trade the close-range monitoring system among them. One user is also allowed to use multiple close-range monitoring systems, for example, use one located in the computer at home, then use one located in the cellular phone when not at home. The close range monitoring system can receive information, analyze, store and provide suggestions to the user (3). Through pictures, images, voice, words, feel, vibration, 3D projections, VR software or electric shocks, the system can guide the user. For example, a VR software located at the close range monitoring system could proved certain tests for the user to determine his or her mental state or intelligence, allowing user (3) to further understand his or her living status. When necessary, the user can communicate with the long-range monitoring station 4 and speak with medical personnel, physical therapists, psychologists or nutritionists to receive instructions to improve the user's situation. For example, the sensors can determine the user's body fat distribution and then have physicians instruct the user on how to exercise to lose weight. After the advice, the system can continuously follow-up to determine if the user performs the exercises properly and burns the proper amount of calories, then test the user's muscular activity and calories burned each day to determine if the proper results are being achieved. Camcorders can be used in all this to determine the user's dietary intake. If necessary, the user can contact the close-range monitoring system or the long-range monitoring station for assistance.

Also, this invention can be used during sleep. While sleeping, the user's eye muscles will move. This allows the invention to monitor and analyze the time the user went to bed, the time it took the user to fall asleep, sleep duration, level of relaxation for different regions of the body, breathing and heart rate changes to determine sleep quality and give suggestions for improvements. For example the suggestion could be medication or behavior therapy, which includes:

A) Sleep hygiene: whether the sleeper could be woken up by calling him or her; does the person sleep regularly; does the person wake up regularly; if the person does not fall asleep after going to bed for twenty minutes, the system may suggest the user to get out of bed and perform various repetitive tasks and go back to bed when the user feels tired.

B) Relaxation therapy: uses images and voice to guide the user to perform progressive muscle relaxations and at the same time follow-up and determine if the user's muscles are relaxing properly; or the system could suggest meditation, imagery or biofeedback to let the user fall asleep more easily. Various VR software could be used to heighten this effect.

C) Stimulus control therapy: by suggesting certain behavior changes, lower the amount of stimulus that could possible wake up the user from sleep to allow the user to sleep better.

D) Chronotherapy: use voice, words, images, feel, vibration and various VR software to adjust the time frame which the user sleeps to match those of a normal time frame.

E) Light therapy: suggest the user to shine a light on his/her face for a duration of time at sunrise or sunset to advance or delay the user's biological clock and then use images, voice, words, feel, vibration, various 3D projections and VR software to guide the user while continuously following-up on the results. If no improvements are made, suggest the user to seek out medical personnel or contact the long range monitoring station (4) for assistance.

Taking things one step further, the monitoring system can use camcorders to provide real time images for the close-range monitoring system to judge the user's status, or it can transmit these images to the long-range monitoring station (4( for analysis. Even the two way communication between the user and the long range monitoring station can utilize these camcorders to transmit images or utilize various messaging systems for two way messaging.

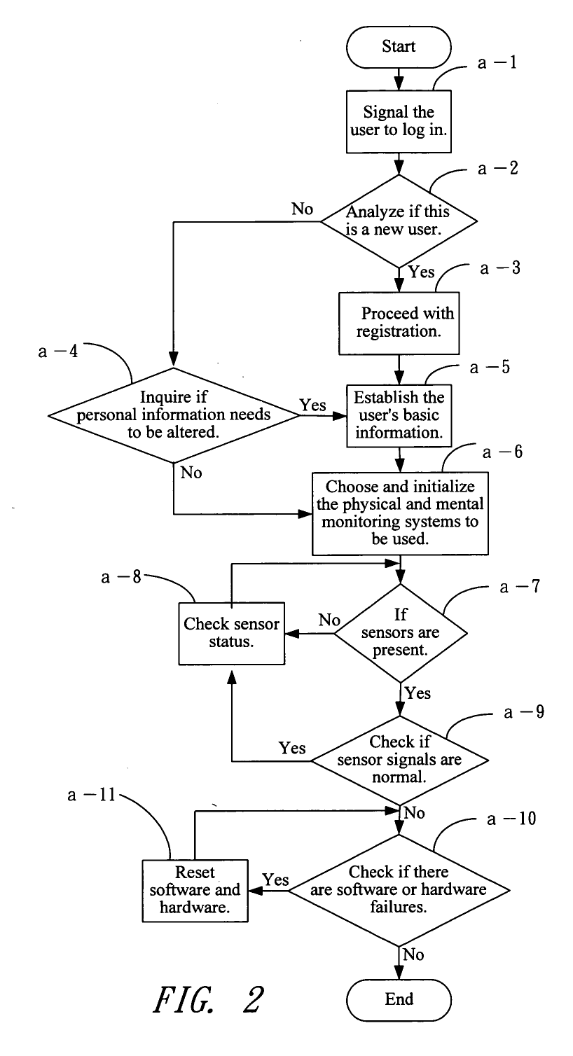

As shown in FIG. 2, this invention's monitoring system requires some prior setup; its procedures are as follows:

a-1) First signaling the user to log in.

a-2) Analyzing if this is a new user.

a-3) If this is a new user, proceed with registration.

a-4) If this is not a new user, inquire if personal information needs to be altered.

a-5) Establish the user's basic information, especially emergency contacts through phone, cell phone, pager, PDA, e-mail, MSN, ICQ, Messenger, et cetera.

a-6) Initialize physical and mental monitoring systems and biomedical profile.

a-7) Check if the sensors are present. If there is no response, then verify sensor's position.

a-8) Check if sensor signals are normal. If not, then verify sensor status.

a-9) Check if other software and hardware are functional. If not, reset software and hardware.

The above procedure will ensure the proper functioning of the system.

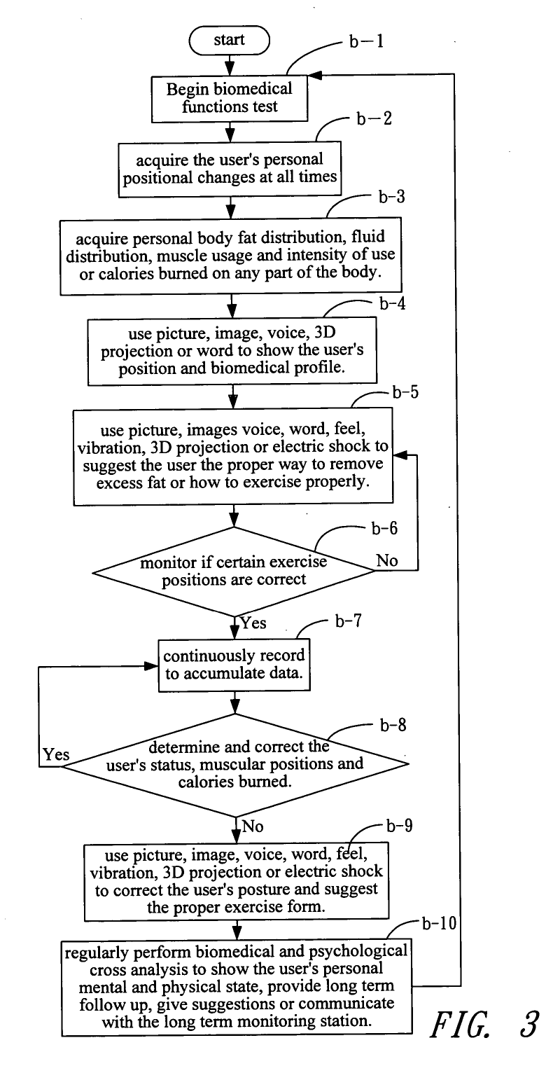

As shown in FIG. 3, the standard functioning procedure of this invention is as follows:

b-1) Initiate biomedical status check.

b-2) Regularly obtain the position of the user to use as a basis for suggestions.

b-3) Obtain information on individual muscle usage and intensity, calories burned, body fat content, fluid content, MEG, heat flow, stress and torque information to use as a basis for suggestions.

b-4) Use picture, image, word, voice or 3D projection to show the biomedical profile of the user along with positional changes to allow medical personnel to better understand the situation.

b-5) Use picture, image, voice, word, feel, vibration, 3D projection or electric shock to suggest the proper exercise form to lose fat in certain areas.

b-6) Monitor if certain exercises are preformed properly, if not, then give new suggestion.

b-7) Continuously record various biomedical information to build a data bank for analysis.

b-8) Use the information described above to analyze the accuracy of personal position changes and the amount of calories burned, if incorrect, then repeat the step before and re-monitor.

b-9) Use picture, image, voice, word, tactile, vibration, 3D projection or electric shock to suggest different body positions, and continuously follow up to correct the user's posture.

b-10) Perform regular biomedical and psychological cross-analysis to determine the user's mental and physical status. Using the depression index to show signs of depression or using the MMSE (Mini-Mental State Examination) intelligence index to show intelligence, or using the Barthel's Score to show personal daily life functions and use drawing software (such as digital white board and paint) to allow the user to express his/her emotions and self evaluate his or her daily functions. The system can suggest the depressed to listen to music, and provide long term follow up. If necessary, contact the psychologist or physician for help.

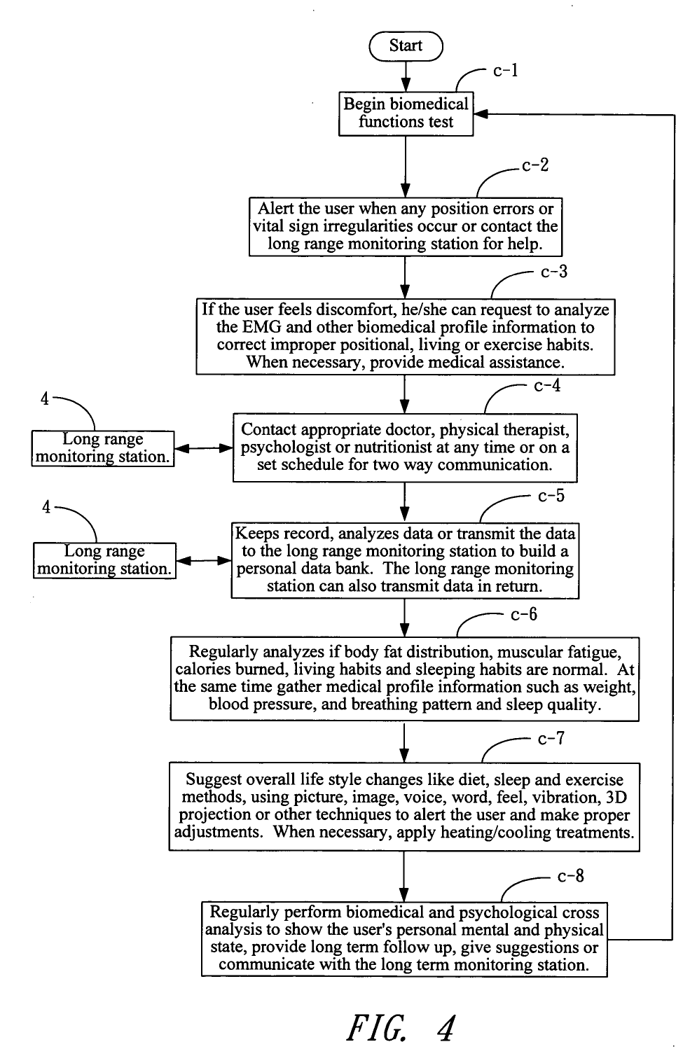

As shown in FIG. 4, this invention can provide more urgent medical assistance; its procedures are as follows:

c-1) Perform biomedical status check;

c-2) When the user experiences certain abnormalities, the system will give warning and suggestions to the user or signal for medical personnel located at the long range monitoring station to give assistance. For example, if the user remains in a certain improper position for too long, the system will inform the user to correct his/her posture. Also, when the user experiences rising blood pressure, difficulty breathing, increased heart rate or dramatic temperature change, the system will warn the user and alert the medical personnel located at the long range monitoring station for help;

c-3) When the user feels discomfort, he/she can use buttons, words or voice to request EMG values and other biomedical profile values be analyzed to find the source of the discomfort. The close range monitoring system will then continuously follow up on the status of the user. When necessary, the system will perform certain medical procedures such as heating/cooling applications, ultra sound or TENS treatment;

c-4) The user can communicate at any time with the personnel located at the long range monitoring station 4 through phone, cellular phone, radio, PDA, computer or internet. This allows the user to receive better medical care as well as allow the personnel located at the long range monitoring station to contact the user when abnormal conditions are detected;

c-5) Maintain a continuous record, analyze information, storing results and transmitting the results to the long range monitoring station (4) to establish a personal biomedical profile for comparison analysis along with blood sugar concentration, cholesterol level, liver functions and kidney functions. The results are transferred back to the close range monitoring system for suggestions;

c-6) Regularly analyzes body fat distribution, muscular activity and calories burned to determine if the user's lifestyle is normal. The system can also analyze the user's weight, blood pressure, breathing and other biomedical information;

c-7) Suggest overall life style changes like diet, sleep and exercise methods, using picture, image, voice, word, feel, vibration, 3D projection or other techniques to alert the user and make proper adjustments. If the user performs certain exercises incorrect and cause muscular inflammation, then the close range monitoring system can signal the garment to auto inflate or allow the user to manual inflate to apply pressure to the proper regions, after a set duration of time, the garment will automatically deflate to prevent side effects from occurring.

Of course, this invention can also be used to monitor various disorders, for example, asthma, lung cancer or other substance abuse disorders. The system can suggest and guild the user through various tasks over a long period of time. In lab tests, after having diagnosed the user who has coughed continuously for two to three month, the close range monitoring system signals the user to get an X-ray examination or to examine the user's mucus. If no improvements are made, then the system checks to see wither the user has lung cancer and sends this information to the long range monitoring station for analysis. If it does turn out to be a certain disorder, then the system will continuously remind the user to take his/her medication. For patients that must be isolated from others, for example: SARS, we can measure the user's biomedical profile and transfer this information to the long range monitoring station to be analyzed. For patients that must be separated from the other patient, for example: SARS patients, the system can monitor their body temperature and transfer any abnormal changes to the long range monitoring station (4) for analysis. When any values are in the abnormal range, the frequency of follow ups will increase and continuously increase to more effectively gather vital information. For example, if body temperature is set to be checked once every twenty minutes and the user's temperature rises abnormally, then the system will increase the frequency to measure body temperature every ten minutes. If there is still no improvements, the system will begin to check every five minutes and eventually every two minutes to acquire more accurate data. For monitoring substance abuse, the close range monitoring system will monitor if such substances are used and signal the long range monitor station (4) if the substance is used. In other words, this invention is not limited to use by normal individuals, it can be used to monitor convicts and patients in isolation as well to prevent any accidents from occurring. This invention can be further modified to monitor animal health, all that needs to be changed is to define the special characteristics of the animal and decide which regions require monitoring.

Some examples of this invention's applications are as follows:

1.) The pregnant mother's physical and mental state along with the contraction rate can be combined to analyze the status of the pregnant mother. At the same time the system could monitor the pulse and movements of the fetus and signal the long range monitoring station if any aid is required.

2.) This invention can be modified to monitor animal daily behavior.

3.) If the user is blind, this invention can use camcorders and other sensors to guide the user through daily tasks.

4.) If the user is deaf, this system can use sound detection sensors to detect sounds and display them using image, word, picture, vibration, feel or 3D projection to the user in the form of language or sign language. If the user is mute, then the system can translate the user's input into sound to communication with others.

5.) If the user is a smoker, this invention could be used to keep record of the amount and frequency in which the user smokes and regularly give warning and suggestions to the user.

6.) The close range monitoring system can also be used to alert the user of various daily tasks like taking medication, injections or using inhalers on a set schedule and follow up on the effects of these tasks and analyze the results.

7.) The close range monitoring system can also be used to alert the medical personnel or family members when psychological disorders occur.

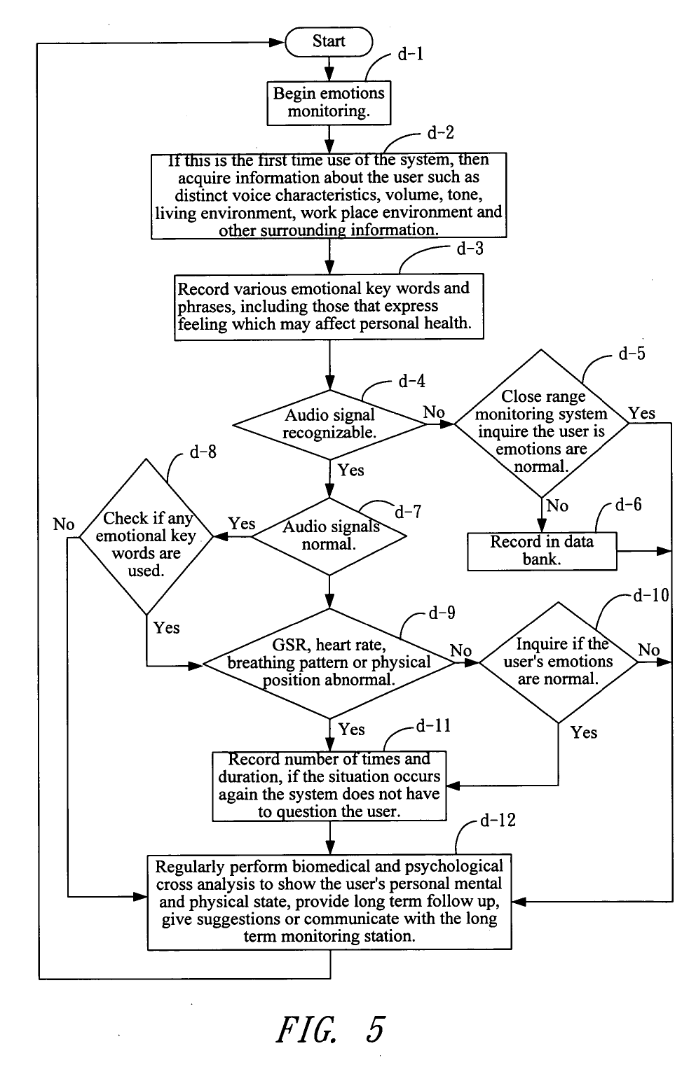

As shown in FIG. 5, the emotional changes of the user can greatly affect the user's health and can not be ignored. For problems that develop as a result of emotional changes, the user can consult a psychologist for advice to prevent accidents from occurring. The procedures for this invention to monitor emotional changes are as follows:

d-1) Initialize emotional monitoring.

d-2) If this is the user's first time using the system, then first acquire information about the user such as distinct voice characteristics, volume, tone, living environment, work place environment and other surrounding information to perform combinational analysis.

d-3) Record various emotional key words and phrases, including those that express feeling which may affect personal health. Also record various key words that express discomfort or various symptoms for disease. Based on voice characteristics of the user, build an emotions data bank.

The initial acquisition of the user's voice can be done voluntarily by the user or at the request of the close range monitoring system (2) or the long range monitoring station (4). Once the emotions data bank is established, depression score, MMSE IQ and other statistics could be acquired through various VR or drawing software.

d-4) Use voice recognition to inquire if the test should be preformed.

d-5) When the audio signal is unclear, then allow the close range monitoring system (2) to inquire if emotions are normal. The close range monitoring system can also acquire camera imaging to aid in the evaluation.

d-6) If emotions are not normal, then distinguishes between happy and unhappy, and record the incident in the data bank;

d-7) If the audio signal is recognizable, then analyze if the voice characteristics are normal.

d-8) If the voice characteristics are normal, then analyze if any emotional key words are used, if no emotional key words are used, then evaluate the user's emotions as being normal.

d-9) If the voice characteristics are not normal, or if any emotional key words are used, then analyze if GSR, heart rate, breathing or body movements are abnormal.

d-10) If GSR, heart rate or breathing patterns are normal, then ask the user if personal emotions are abnormal, if yes, record in data bank. The close range monitoring system can also acquire camera imaging to aid in the evaluation.

d-11) If GSR, heart rate or breathing patterns are not normal, then record in data bank and also record the number of times these abnormal emotions occur and the duration that they occur for. In the future, if similar problems occur, the close range monitoring system will not need to ask all these questions again.

d-12) After the above procedure determines if emotions are normal or abnormal, the system can regularly perform physical and mental analysis to determine the user's health status, depression index, intelligence level and various other statistics. The close range monitoring system will give suggestions to the user based on the result of this analysis. If necessary, physicians, physical therapists or psychologists could be contacted for two way communication.

To deal with noisy environment, poor sleep quality and any other emergencies that may happen, this invention has the following procedure:

a. Record the duration of time each day that that user is in a noisy environment, and suggest the user to improve on this situation.

b. Record the talk time, volume and tone of the user each day and use word, voice, image, 3D projection or vibration to suggest the user various pronunciation or breathing techniques, if no improvements are made, then contact the medical personnel at the long range monitoring station for help. If the user's breathing stops during sleep, then record and use voice, electric shock or vibration to alert the user.

c. Special key words such as “suicide” or “die” along with not speaking for a long period of time, falling, crying, screaming and other abnormal behavior could cause the system to immediately contact the long range monitoring station (4).

d. The amount of food ingested and the duration of time ingesting could be monitored through EMG or sound, when the signal is unclear the system could directly ask the user.

e. Sleep statistics can be analyzed through posture, breathing, heart rate, various muscular relaxation levels, the time it takes to fall asleep, sleep duration, bed time and other information. The system will give suggestions based on the analysis and suggest things such as medication or behavior therapy and continuously follow up and analyze if improvements are made. If no improvements are made, then the system can contact the long range monitoring station for two way communication.

f. Through an analysis of the user's breathing, the system could determine if the user has COPD, asthma attack or emphysema attack. If yes then the system will suggest the proper medication, if the situation does not improve, then the long range monitoring station could be contacted for two way communication.

g. Through various VR or drawing software, the user can express his/her emotional and intelligence status or self evaluate his/her daily functions.

The procedure mentioned above can determine the user's emotional state and alert the user of his/her state. It can also suggest to the user various treatments such as music, TENS, ultrasound, heating/cooling applications or to contact the long range monitoring station for assistance. Many of these applications could be installed directly on to the garment. When the depression score is too high, or when the user expresses that he/she wants to speak with someone, the close range monitoring system could use 3D projections of live or VR images to interact with the user, when necessary, the personnel located at the long range monitoring station (4) can perform live conferencing with the user to quickly alleviate depression and prevent the occurrence of accidents. When needed, specialists and/or pets could be dispatched to the user. There are many long range monitoring stations, and the user can choose the most appropriate one for service, when necessary, multiple long range monitoring stations can combine efforts to find the best solution. Using camera imaging, the long term monitoring station (4) can also attempt to determine the user's status through facial expressions and present the proper assistance.

This invention will help pregnant mothers maintain their health, through constant monitoring and suggestions, the system will let the pregnant mother know the best exercise to perform. The system also monitors the movement and pulse of the fetus to ensure its health. This invention also helps the mother to slim down after giving birth. This invention can also monitor people that are diabetic, over weight, depressed or have other disorders. If necessary, it will contact the long range monitoring station for two way communication. The close range monitoring system can also be used to monitor the user's environment. For example, if the temperature indoors is too high during sleep, the system could lower the air conditioning; or if there is a gas leak in the house, the system could alert the user.

### INDUSTRIAL APPLICATIONS

This invention provides means of monitoring and analyzing the personal physical and mental health status. This invention can scientifically monitor physical and emotional states of the user for analysis, and this invention will continuously follow up to promote a healthier living. By analyzing and suggesting various health tips and precautions, this invention can help create a healthier society. This invention can also be modified to monitor the health of animals for analysis.

Many changes and modifications in the above described embodiments of the invention can, of course, be carried out without departing from the scope thereof. Accordingly, to promote the progress in science and the useful arts, the invention is disclosed and is intended to be limited only by the scope of the appended claims.

What is claimed is:

1. A physical and mental health status monitoring, analyzing, and automated response system, utilizing:
a) A close range monitoring system with microprocessors using one or multiple sensors located in the user's cloth to acquire personal behavior and posture change information at any given time.
b) The close range monitoring system can also acquire personal body fat distribution, heat flow, fluid distribution, muscle usage and intensity of use, calories burned, pressure or torque exerted on any part of the body.
c) The close range monitoring system can use picture, image, voice, 3D projection or word to show the user's health status and biomedical profile. This allows the user to know about his/her body health.
d) The close range monitoring system can use picture, images voice, word, feel, vibration, 3D projection or electric shock to suggest the user the proper way to remove excess fat or how to exercise the proper region. It also monitors if these exercises are done properly and correct the user's posture when not done properly.
e) The close range monitoring system keeps records and analyzes these records to regularly report. If necessary, the user can contact the long range monitoring station according to the result of these reports for two way communication.
f) A long range monitoring station can judge and correct the user's status, muscular positions and calories burned based on information provided by the close range monitoring system.
g) The long range monitoring station can use picture, image, voice, word, feel, vibration, 3D projection or electric shock to correct the user's posture and suggest the proper exercise form. It also continuously follows up to provide any advice if needed. The long range monitoring station can contact the user or be contacted by the user for information.

2. As stated in claim 1, this invention also includes:
a) The close range monitoring system will alert the user when any position errors or vital sign irregularities occur and can contact the long range monitoring station for help if necessary.
b) If the user feels discomfort, he/she can use buttons, words or voice to request the close range monitoring system to analyze the EMG and other information to locate the source of the discomfort and suggest proper procedure to ease this discomfort.
c) Using the long range monitoring station, the user can contact appropriate doctor, physical therapist, psychologist or nutritionist at any time or on a set schedule for two way communication.
d) The close range monitoring system keeps record, analyzes data, shows results to the user, contacts long range monitoring station data banks and uses a combination of other health information to improve personal health status through communication with medical personnel, physical therapist or nutritionist.
e) The close range monitoring system periodically analyzes if body fat distribution, muscular fatigue, calories burned, hiving habits and sleeping habits are normal.
f) The close range monitoring system periodically provides the user advice, and using picture, image, voice, word, feel, vibration, and 3D projection to tell the user the proper method and positions to achieve proper exercise or relaxation. The monitoring system also keeps following up and analyzing to provide more advice.
g) If the close range monitoring system encounters emergencies such as falls, contractions or heart/breathing failures, it can immediately send signals for the long range monitoring station to provide further actions.

3. As stated in claim 1, this invention also includes:
a) On the first use, the close range monitoring system first analyzes the user's voice pattern, including volume, tone and range.
b) The close range monitoring system records some of the key words, including any emotional or health status effecting terms.
c) After acquiring the user's voice, the user can program the system to receive information from the long range monitoring station or the close range monitoring system to build personal emotions data bank, acquire depression symptoms chart, MMSE intelligence quotient chart, et cetera. The method of acquisition may include digital drawing software (such as digital white board or paint) or other VR software for the user to show his/her emotional status.
d) The close range monitoring system can determine the current status of the user's voice.
e) When the voice input is not recognizable, the close range monitoring system will ask the user if emotions are normal, if not, it will record in the data bank. The close range monitoring system can also acquire camera imaging to aid in the evaluation.
f) If voice input is normal, the close range monitoring system will determine if any emotional key words are used, if not, then the procedure will end.
g) If voice input is not normal, or if emotional key words are used, then the close range monitoring system will further analyze GSR, heart rate, breathing, and body movements to determine the user's status.
h) If GSR, heart beat, breathing or body movements are irregular, then the close range monitoring system records down the number of times repeated and duration.
i) If GSR, heart beat and breathing are normal, then the close range monitoring system will inquire if emotions are normal, if not, then it will record in the data bank. The close range monitoring system can also acquire camera imaging to aid in the evaluation.
j) The close range monitoring system will record the number of emotional irregularities, duration and time. After data is established, if the same situation occurs at a later time, the system will not have to repeat the same questions.
k) The close range monitoring system regularly performs physical and mental cross-analysis to show the user's personally health status. For example: a depression meter shows how depressed the user is; MMSE (intelligence quotient chart) shows intelligence status; stress and relaxation level or Barthe's Score show personal daily life functions; and drawing software (such as the digital whiteboard or paint), et cetera allow the user to express his or her emotions, intelligence level, and self-evaluate daily life functions. It can also give advice. For example: it will tell depressed people to listen to music; it will tell people with low IQs to train their brain; and if the user needs to, the user can communicate with psychologists, doctors, and physical therapists at the long range monitoring station.

4. As stated in claim 1, the close-range monitoring system's functioning procedure includes:
a) First signaling the user to log in.
b) Analyzing if this is a new user.
c) If this is a new user, proceed with registration.
d) If this is not a new user, inquire if personal information needs to be altered.
e) Establish the user's basic information, especially emergency contacts through phone, cell phone, pager, PDA, e-mail, MSN, ICQ, Messenger, et cetera.
f) Initialize physical and mental monitoring systems.
g) Check if the sensors are present. If there is no response, then verify sensor's position.
h) Check if sensor signals are normal. If not, then verify sensor status.
i) Check if other software and hardware are functional.
If not, reset software and hardware.

5. As stated in claim 1, the close-range monitoring system also records the duration, the volume and percentage of total time monitored in this environment to suggest to the user improvements and follow-ups on the diagnosis.

6. As stated in claim 1, the close-range monitoring system also records the user's total talk time, volume, and changes in tone to give suggestions in pronunciation and breathing exercises. It will then follow-up on the results of the suggestions and it can allow two-way communication with medical personnel located at the long-range monitoring station.

7. As stated in claim 1, the close-range monitoring system also compares the user's physical and mental data with the standard data for people of the same age to analyze and display suggestions.

8. As stated in claim 1, the close-range monitoring system also uses the user's change in life-style and mood swings to analyze if there is are symptoms for degeneration of memory and gives suggestions and guidelines.

9. As stated in claim 1, the close-range monitoring system uses the user's daily amount of exercise, body changes (temperature, sweat, and heat flow) to determine the amount of calories needed, suggest personal nutrition, and recommend exercise routines. The system also uses a camcorder to monitor the food content and further suggest if various foods should be eaten and continuously follows-up and monitors to maintain a slim figure.

10. As stated in claim 1, the close-range monitoring system reminds the user of various important daily tasks such as taking medication, injections, and using an inhaler. It also continually follow-ups and analyzes if improvements are being made. If no progress is found, the system can signal the long-range monitoring station for two-way communication.

11. As stated in claim 1, the close-range monitoring system judges sleep quality through when the user goes to bed, time it takes the user to fall asleep, heart beat, breathing pattern, muscular relaxation level, number of times the user gets up at night, and the duration of sleep. If sleep quality is poor, the system will suggest medication and behavior therapy. Then the system will continuously monitor, follow-up, and analyze if there is any improvement. If no progress is found, the system can signal the long-range monitoring station for two-way communication.

12. As stated in claim 1, the close-range monitoring system also analyses living habits, emotional changes, and living environment together to determine emotional and physical health changes. If negative changes occur, it will give warnings and suggestions. When necessary, the user can contact the long-range monitoring station for two-way communication.

13. As stated in claim 1, the close-range monitoring system also monitors various muscle groups and regions of fat to determine if there is atrophy or thickening of fat. It will also check to see if any nerve endings atrophied or are over reactive. The system will also give rehabilitation advice. Then the system will continuously monitor, follow-up, and analyze if there is any improvement. If no progress is found, the system can signal the long-range monitoring station for two-way communication.

14. As stated in claim 1, the close-range monitoring system also analyzes breathing pattern, muscle usage, heart beat, sweat, emotional change, calories burned, stress level and torque level on various parts of the body to determine if the user's body can adapt to exercise properly. This will regularly provide the user with analysis results and suggestions. Then the system will continuously monitor, follow-up, and analyze if there is any improvement. If no progress is found, the system can signal the long-range monitoring station for two-way communication.

15. As stated in claim 1, the close-range monitoring system can determine through various voice patterns if the user has asthma or COPD or Emphysema. The system will also give suggestions and if no improvements are made, it can signal the long-range monitoring station for two-way communication.

16. As stated in claim 1, the close-range monitoring system can monitor the pregnant mother's physical and mental status, contraction rate, fetal movement and fetal pulse to determine the pregnant mother's health status and whether the contraction rate is normal. The system will also give suggestions and if necessary, it can signal the long-range monitoring station for two-way communication.

17. As stated in claim 1, the close-range monitoring system can be programmed to monitor different information and can be used on animals to determine its living habits.

18. As stated in claim 1, the close-range monitoring system can use cameras and other detectors to guide the user through daily tasks if the user is blind.

19. As stated in claim 1, the close-range monitoring system can use sound detection devices and through pictures, words, images, feel, vibration, 3-D projections, et cetera to display various languages and sign languages. It can automatically translate this into voice to communicate to the outside world if the user is deaf.

20. As stated in claim 1, if the user is a smoker, the close-range monitoring system can record the amount and frequency the user smokes and give regular warnings and suggestions. If no improvements are made, it can signal the long-range monitoring station for two-way communication.

21. As stated in claim 1, the close-range monitoring system can automatically inflate or allow the user to manually inflate when various exercises and positions cause muscle inflammation. After a set duration of time, it will automatically deflate or tell the user to manually deflate to prevent any side affects.

22. As stated in claim 1, the close-range monitoring system can provide pictures, music, words, voice, and 3-D projections of virtual reality or live people to interact with the user when the user is depressed or requests to speak with another individual through the system. When necessary, it can signal the long-range monitoring station to request video conferencing to alleviate depression or send specialists, even pets to lower the chance of accidents.

23. As stated in claim 1, if sleep quality is poor, the close-range monitoring system can provide corrective suggestions; including sleep hygiene, relaxation therapy, stimulus control therapy, or other therapies. Through images, voice, words, 3-D projections, virtual reality software, feel, vibration, et cetera, it can give suggestions to the user. The system will continuously monitor and follow-up and if no improvements are made, it will suggest to seek medical personnel or transfer to the long-range monitoring station for assistance.

24. As stated in claim 1, the user can interact with the long-range monitoring station through various messaging services and use camcorder functions to achieve better interaction.

25. A physical and mental health status monitoring, diagnosing, and follow-up clothing system including:
a) Sensors placed on various parts of the clothing to acquire personal health data
b) Close-range monitoring systems to receive, analyze, store, and transmit data from the sensors or the long-range monitoring station. Using pictures, voice, images, words, feel, vibration, 3-D projections, drawing software, and virtual reality software to provide the proper personnel information. Based on the results, automatically follow-up and repeatedly monitor.
c) Long-range monitoring stations receive and transfers data to and from the close-range monitoring system.

26. As stated in claim 25, the sensors can be placed within cloth, hat, glove, sock, belt and various other forms of clothing and be directly worn by the user.

27. As stated in claim 25, the sensors can be stationary or removable and placed on cloth, hat, glove, sock, belt and various other forms of clothing. The sensors can also be programmed to move on their own at the request of the user or the close range monitoring system to gather data from different areas of the body.

28. As stated in claim 25, the sensors consist of a group of audio sensors, breathing sensors, temperature sensors, ultra sound sensors, position sensors, pressure sensors(for pressure and torque), blood oxygen concentration sensors, blood pressure sensors, motion sensors, heat sensors, et cetera.

29. As stated in claim 25, the close range monitoring system can also be placed directly within the users clothing or be separately positioned in PDA, computer, cellular phone or other devices; the close range monitoring system's functions can be customized by the user to include GPS, GPRS, LEOS, 3G, ECDS, MMS, GSM, CDMA, Blue Tooth or MPS.

30. As stated in claim 25, the sensors can also be implanted in the user's body.

31. As stated in claim 25, the close range monitoring system can use camcorders to obtain images of the user's living habits for the long range monitoring station to analyze.

32. As stated in claim 25, the close range monitoring system can analyze the user's sleep duration and quality over a long period of time. If the user stops breathing, the close range monitoring system will record the incident and signal, vibrate or shock the user to stimulate the user. If necessary, it will also signal the personnel at the long range monitoring station for assistance.

33. As stated in claim 25, the clothing system can also have massaging devices (TENS), ultra sound devices or heating/cooling devices installed.

34. As stated in claim 25, there may be multiple long range monitoring stations and the user can choose which long range monitoring station to contact for assistance. When necessary, the different long range monitoring stations can work together to find the best solution.

35. As stated in claim 25, the sensors can be placed in bathtubs, mattresses, bed frames, toilets, wheel chairs, chairs and other locations to gather data when the user has to remove his/her clothing or when the user is in a location where clothing is not worn.

36. As stated in claim 25, the clothing system can have illumination devices or fiber optics installed. When necessary, these lighting devices can display words or pictures to signal the user or other personnel.

37. As stated in claim 25, bar codes can be placed on items that the user regularly comes in contact with, bar code readers can be placed on the user to regulate the number of times the user uses such an item. This way, the system can monitor excessive use of various items to prevent accidents. The bar codes mentioned above can be replaced by electronic tags and the bar code reader can be replaced by the RFID system.

38. As stated in claim 25, grids can be installed on the walls at the user's home. This grid can have camera (CMOS or CCD ) or heat imaging devices installed to monitor the user's position within the house at a given time. If the user is in a place where he/she normally does not visit at a given time, the system will evaluate the user's actions to determine if there are any accidents. The camera imaging devices can continuous monitoring of the user's facial expressions to help determine the user's emotions at a given time.

39. As stated in claim 38, the sensors and the identification procedures can use the RFID (Radio Frequency Identification) method.

40. As stated in claim 25, if various values in the biomedical profile become abnormal, the follow up frequency will increase, and if the values remain abnormal, the frequency will further increase to gather data for analysis.
